(19) United States
(12) Patent Application Publication
(10) Pub. No.: US 2014/0316246 A1
(43) Pub. Date: Oct. 23, 2014

(54) CHEMICAL EXCHANGE SATURATION TRANSFER ANGIOGRAPHY

(71) Applicant: University of Massachusetts Medical School, Boston, MA (US)

(72) Inventors: Matthew J. Gounis, Shrewsbury, MA (US); Shaokuan Zheng, Northborough, MA (US); Imramsjah Martijn John van der Bom, Boston, MA (US)

(21) Appl. No.: 14/258,912

(22) Filed: Apr. 22, 2014

Related U.S. Application Data
(60) Provisional application No. 61/815,235, filed on Apr. 23, 2013.

(51) Int. Cl. A61K 49/06; A61B 5/055

(52) U.S. Cl. CPC A61K 49/06; A61B 5/055
USPC 600/410; 424/9.3

(57) ABSTRACT

The invention provides a novel approach for effective ex vivo and in vivo imaging of blood and establishes the feasibility of blood as an effective agent for CEST to generate sufficient CEST contrast relative to surrounding tissue.

### PRIORITY CLAIMS AND RELATED APPLICATIONS

This application claims the benefit of U.S. Provisional Application No. 61/815,235, filed Apr. 23, 2013, the entire content of which is incorporated herein by reference in its entirety.

### TECHNICAL FIELD OF THE INVENTION

The invention generally relates to methods and compositions for molecular resonance imaging. More particularly, the invention relates to a novel technique and related methods and compositions for Chemical Exchange Transfer Saturation (CEST)-based in vivo imaging of blood wherein blood is employed as an agent for effecting CEST.

### BACKGROUND OF THE INVENTION

CEST imaging, an emerging magnetic resonance (MR) imaging contrast technique, has paved the way for a variety of novel clinical applications. (Ward, et al. 2000 J Magn Reson. 143:79-87.) In CEST, exogenous or endogenous compounds containing exchangeable protons with water protons are selectively saturated and after transfer of this saturation, detected indirectly through the water signal with enhanced sensitivity. When the exchange rate is sufficiently fast (but in the slow or intermediate exchange regime on the NMR time scale) and the saturation time is sufficiently long, enough bulk water protons will be saturated and the water signal will be attenuated. As a result, contrast enhancements up to 500,000 can be achieved for certain chemical compositions. (Goffeney, et al. 2001 J Am Chem Soc. 123:8628-8629; Zhou, et al. 2006 Prog Nucl Mag Res. 48:109-136.) CEST thus provides a new mechanism for indirect detection of dilute labile protons that are normally invisible by conventional MRI. (van Zijl, et al. 2011 Magn Reson Med. 65:927-948.)

For example, WO 2000/066180 describes a method for enhancing contrast produced in MRI by performing CEST. The image contrast is obtained by altering the intensity of the water proton signal (by selectively saturating a pool of exchangeable protons of the CEST contrast agent, by using an RF pulse). These protons subsequently transfer the saturation to nearby water by exchange with water protons, thereby decreasing the water proton signal. WO 2000/066180's procedure includes: selecting one or more contrast agents; administering same or a composition containing same to a subject; irradiating, and thereby saturating, (exchangeable) protons of the contrast agent at a first predetermined frequency (+Δω_{cA}) of the water proton signal and providing an image; irradiating at a second predetermined frequency (−Δω_{cA}), which may also be called off-resonance, of the water proton signal and providing a second image; and determining a third image provided by the subtraction or ratio of the first image relative to the second image, (±Δω_{cA}) referring to the chemical shift difference between the resonance of an exchangeable entity of a contrast agent and the water proton resonance.

CEST has been exploited for various applications, such as the imaging of brain tumors, cartilage in joints, intervertebral discs, muscle, and liver. (Jones, et al. 2006 Magn Reson Med. 56:585-592; Ling, et al. 2008 Proc Natl Acad Sci USA. 105:2266-2270; Schmitt, et al. 2011 Radiology. 260:257-264; Kim, et al. 2011 NMR Biomed. 24:1137-1144; Saar, et al. 2012 NMR Biomed. 25:255-261; van Zijl, et al. 2007 Proc Natl Acad Sci USA. 104:4359-4364.) In addition, it has been proposed to use CEST to measure pH-values (both ex vivo and in vivo), enzymatic activity, and temperature. (Zhou, et al. 2003 Nat Med. 9:1085-1090; Ward, et al. 2000 Magn Reson Med. 44:799-802; Aime, et al. 2002 Angew Chem. 114:4510-4512; Aime, et al. 2002 Magn Reson Med. 47:639-648; Yoo, et al. 2006 J Am Chem Soc. 128:14032-14033; Zhang, et al. 2005 J Am Chem Soc. 127:17572-17573.)

In these applications, endogenous agents (e.g., amide protons (—NH) from proteins and peptides, amine protons (—NH₂) from glutamate, and hydroxyl protons (—OH) from glycosaminoglycan (GAG) and glycogen are used, which are naturally present in vivo. (Jones, et al. 2006 Magn Reson Med. 56:585-592; Ling, et al. 2008 Proc Natl Acad Sci USA. 105:2266-2270; Schmitt, et al. 2011 Radiology. 260:257-264; Kim, et al. 2011 NMR Biomed. 24:1137-1144; Saar, et al. 2012 NMR Biomed. 25:255-261; van Zijl, et al. 2007 Proc Natl Acad Sci USA. 104:4359-4364; Zhou, et al. 2003 Nat Med. 9:1085-1090. Cai, et al. 2012 Nat Med. 18:302-306.) In addition, reports have shown that exogenous paramagnetic agents can be used to enhance the CEST effect and to overcome the limitation of slow or intermediate exchange. (Zhou, et al. 2006 Prog Nucl Mag Res. 48:109-136; van Zijl, et al. 2011 Magn Reson Med. 65:927-948; Aime, et al. 2002 Magn Reson Med. 47:639-648; Aime, et al. 2002 J Am Chem Soc. 124:9364-9365; Woessner, et al. 2005 Magn Reson Med. 53:790-799; Zhang, et al. 2003 Acc Chem Res.;36:783-790; Zhang, et al. 2003 J Am Chem Soc. 125:15288-15289.)

MR imaging of blood in vivo remains a major challenge in part because blood flow affects the intensity of MR images in many nonlinear ways that depend both on details of the particular imaging technique and on blood vessel and flow geometry.

It is strongly desired that novel approaches and techniques are developed that provide effectively in vivo imaging of blood.

### SUMMARY OF THE INVENTION

The invention provides a novel approach for effective ex vivo and in vivo imaging of blood and establishes the feasibility of blood as an effective agent for CEST to generate sufficient CEST contrast relative to surrounding tissue. The method of the invention is based on the measurement of the labile protons that are associated with various amino acids, proteins, peptides and other molecules that are naturally present in blood. These components are not detectable with currently available clinical MRI sequences, but generate a clear signal by using CEST. The disclosure demonstrates that CEST can be used effectively to image blood in vivo.

The CEST effect of porcine blood samples was investigated on a 3.0 T MRI scanner using various power levels and on a 14.1 T NMR spectrometer. Additionally, CEST was effectively used to image blood in vivo when applied in locations of healthy human volunteers (e.g., the femoral artery and the M1-segment of the middle cerebral artery). The ex vivo experiments showed that maximum CEST Magnetization Transfer Ratio asymmetry (MTR_{asym}) values of approximately 12% were achieved. The in vivo experiments showed that CEST signal of blood was significantly greater than surrounding muscular and brain tissue.

In one aspect, the Invention generally relates to a method for imaging blood of a mammal, including human, in vivo. The method includes imaging the mammal using a CEST-based magnetic resonance imaging procedure wherein blood of the subject serves as the CEST contrast agent.

In another aspect, the invention generally relates to a method for performing angiography of a mammal, including human. The method includes performing CEST imaging with blood serving as the endogenous CEST contrast agent.

In yet another aspect, the invention generally relates a to method for diagnosing a mammal, including human, of a disease or condition. The method includes: performing CEST imaging with the mammal's blood serving as the endogenous CEST contrast agent; and obtaining a magnetic resonance image of at least a part of a vascular system of the mammal.

### BRIEF DESCRIPTION OF THE DRAWINGS

FIG. 1. Representative data of the four of the cylindrical tubes with porcine blood samples and ACD emerged in a beaker containing a copper sulfate solution performed on a 3.0 T scanner at room temperature. Corresponding proton density image (a, TR/TE=3000 ms/10 ms); proton CEST MTR_{asym} image at 2.5 ppm corrected for BO using the WASSR method (b), WASSR shift image (c), and matching Z-spectra (d) and MTR_{asym} graphs (e) from whole blood (WB), plasma (PL), red blood cells (RC) and ACD solution.

FIG. 2. Representative Z-spectra and corresponding MTR_{asym} graphs of porcine whole blood, plasma, and red blood cells performed on a 3.0 T scanner at room temperature. Z-spectra (a) and corresponding MTR_{asym} graphs (b) at different pre-saturation power levels are shown for whole blood (1.05 μT, 1.4 μT, 2.1 μT, 2.8 μT, 3.5 μT, 4.2 μT and 5.6 μT). Similar Z-spectra and corresponding MTR_{asym} graphs are given for plasma (c, d), red blood cells (e, f), and ACD (g, h).

FIG. 3. The influence of temperature on the CEST effect in blood samples performed on a 3.0 T scanner. The Z-spectrum and the corresponding MTR_{asym} for the porcine whole blood (a, b), plasma (c, d), and red blood cells (e, f) at room temperature and a physiological temperature of 37° C.

FIG. 4. Results of high-resolution proton NMR spectroscopy experiments without and with water pre-saturation of whole blood (a, b), plasma (c, d), and red blood cells (e, f) using 14.1 T Varian spectrometer at room temperature. Graphs show proton NMR spectra without (a, c, and e) and with water saturation (b, d, and f), and NMR spectrum of ACD with water saturation (g).

FIG. 5. The Z-spectra (a) and the corresponding MTR_{asym} graphs (b) of whole blood, plasma, and red blood cells performed on a 14.1 T spectrometer at room temperature. pp FIG. 6. An example of the in vivo CEST results from a volunteer's leg performed on a 3.0 T scanner. Corresponding anatomic proton image of the thigh with two ROIs placed in the lumen of the femoral artery (arrows) and muscle (a), the CEST image at 3 ppm with WASSR correction (b), WASSR shift image (c), T2w-TSE image (d), and TOF image (e) are given. The Z-spectra (f) and the corresponding MTR_{asym} (g) for the ROIs shown in (a).

FIG. 7. An example of the in vivo CEST results from a human brain performed on a 3.0 T scanner. Corresponding anatomic proton image including the M1-segment (arrows) of the MCA (a), the CEST MTR_{asym} image at 3 ppm with WASSR correction (b), WASSR shift image (c), T2w-TSE image (d), and TOF image (e) are given. The Z-spectra (f) and the corresponding MTR_{asym} (g) for the ROIs located in arterial blood and brain tissue as indicated in (a).

### DETAILED DESCRIPTION OF THE INVENTION

The invention provides a novel approach for effective ex vivo and in vivo imaging of blood and establishes the feasibility of blood as an effective agent for CEST to generate sufficient CEST contrast relative to surrounding tissue.

This disclosure herein demonstrates that CEST can be used effectively to image blood in vivo. The method of the invention is based on the measurement of the labile protons that are associated with various amino acids, proteins, peptides and other molecules that are naturally present in blood. (Krebs 1950 Ann Rev Biochem. 19:409-430; Nicholson, et al. 1995 Anal Chem. 67:793-811.) These components are not detectable with currently available clinical MRI sequences, but generate a clear signal by using CEST.

Magnetic resonance angiography (MRA) is a well-established imaging technology and is widely used for diagnosis of vascular pathologies and treatment follow-up. Because MR imaging (MRI) does not use ionizing radiation, this modality may often be preferred over computed tomography angiography (CTA). Currently available MRA techniques can roughly be classified into three groups: Time-Of-Flight (TOF), Phase-Contrast (PC), and contrast-enhanced (CE) sequences. Because blood signal intensity in TOF and PC MRA are realized by inflow of unsaturated blood into the partially saturated imaging volume and phase changes by blood velocity, respectively, signal loss as a result of different flow patterns can impede the accurate diagnosis of vascular pathologies. For instance, in a simple laminar flow pattern, blood velocity in the center of a vessel will be higher than that adjacent to the vessel wall, which result in a brighter appearance in the center of the vessel than near the vessel wall. Similar artifacts occur as a consequence of (partial) in-plane flow, fast or slow flow, flow stasis, and flow reversal where insufficient unsaturated blood protons enter the imaging volume in order to achieve enough signal-to-noise ratio (SNR) to distinguish blood from its surroundings. Flow artifacts caused by slow or complex flow have shown to occur in (intracranial) aneurysms, where reliable detection of aneurysms and residual flow/regrowth by TOF MRA was limited by loss of blood signal. (Bernstein, et al. 2001 Magn Reson Med. 46:955-962; Deutschmann, et al. 2007 AJNR Am J Neuroradiol. 28:628-634; Boulin, et al. 2001 Radiology. 219:108-113; Cottier 2003 AJNR Am J Neuroradiol.; 24:1797-1803; Gibbs, et al. 2005 JMRI J Magn Reson Im. 21: 97-102; Jäger, et al. 2000 AJNR Am J Neuroradiol. 21:1900-1907; Kaufmann, et al. 2010 AJNR Am J Neuroradiol. 31:912-918; Okahara, et al. 2002 Stroke. 33:1803-1808.) Similar flow artifacts in TOF data are observed as a result of vascular stenosis, which may lead to overestimation of the degree of stenosis and in follow up imaging of (brain) arteriovenous malformations (AVMs), which can result in overestimation of nidus obliteration. (Babiarz, et al. 2008 AJNR Am J Neuroradiol. 30:761-768; Nederkoorn, et al. 2002 AJNR Am J Neuroradiol. 23:1779-1784; Oelerich, et al. 1998 Neuroradiol. 40:567-573; Yang, et al. 2002 AJNR Am J Neuroradiol. 23:557-567; Buis, et al. 2011 AJNR Am J Neuroradiol. 33:232-238.)

The invention disclosed herein demonstrates that that CEST can be used as a non-invasive MR-angiography technique, the first report on the implementation of CEST for MR angiography. Unlike currently available MR angiography techniques, the proposed method is based on the measurement of the labile protons which are associated with various amino acids, proteins, peptides and other molecules that are naturally present in blood. These components are not detectable with currently available clinical MRI sequences, but generate a clear signal by using CEST. It is hypothesized that angioCEST may provide enhanced image quality of vasculature relative to TOF and PC methods for vascular anomalies such as aneurysms and atherosclerotic disease, as it is less susceptible to artifacts caused by pathological or complex flow.

Ex vivo and in vivo experiments below show that blood is a suitable CEST agent that generates sufficient CEST contrast relative to surrounding tissues.

The NMR experiments without water pre-saturation showed two distinguished broad peaks centered at 3 ppm and −3.5 ppm relative to water resonance frequency. It should be mentioned here that these two broad peaks correspond to mobile macromolecules and not to the macromolecular semi-solid lattice since the NMR spectra of the macromolecular semi-solid lattice are too extensive to be visualized. (van Zijl, et al. 2011 Magn Reson Med. 65:927-948; van Ziji, et al. 2003 Magn Reson Med. 49:440-449.) Comparing the NMR results of blood samples without water pre-saturation to those with water pre-saturation, it can be seen that the two distinguishable wide peaks decrease greatly relative to the citric acid peaks at −2.1 ppm relative to water resonance. These results demonstrate that the protons in these frequency ranges interact with water protons. Similar results, obtained by experiments using a 500 MHz NMR spectrometer, were reported in the literature. (Rabenstein, et al. 1988 Anal Chem. 60:A1380-A1391; Rabenstein, et al. 1980 J Magn Reson. 41:361-365.) This phenomenon was explained as the transfer of saturation from protons of macromolecules with a resonance frequency similar to that of water, to other protons related to the same molecules by cross relaxation. It, however, did not clarify the proton transfer between macromolecules and water.

As reviewed in literature, MT between different protons attached to macromolecules and between protons of macromolecules and free water is a common phenomenon in MR spectroscopy and imaging. (Zhou, et al. 2006 Prog Nucl Mag Res. 48:109-136; van Zijl, et al. 2011 Magn Reson Med. 65:927-948; Henkelman, et al. 2001 NMR Biomed. 14:57-64.) Two main mechanisms responsible for MT are chemical exchange and dipolar cross-relaxation. However, CEST is specifically defined as the MT phenomena based on the chemical exchange between solute labile protons and the bulk water protons. In the case of blood, the broad peak at 3 ppm relative to water resonance most likely corresponds to amide (—NH) and amine protons (—NH₂), which have a chemical shift of approximately 3.6 ppm and 2.2 ppm downfield from bulk water, respectively. (Zhou, et al. 2003 Nat Med. 9:1085-1090; Cai, et al. 2012 Nat Med. 18:302-306; Liepinsh, et al. 1996 Magn Reson Med. 35:30-42.) Furthermore, proton exchange between amide protons, amine protons, and water protons were reported with exchange rates (k_{sw}) in the range of 10-140 s⁻¹ and 500-10,000 s⁻¹, for amide protons and amine protons, respectively.

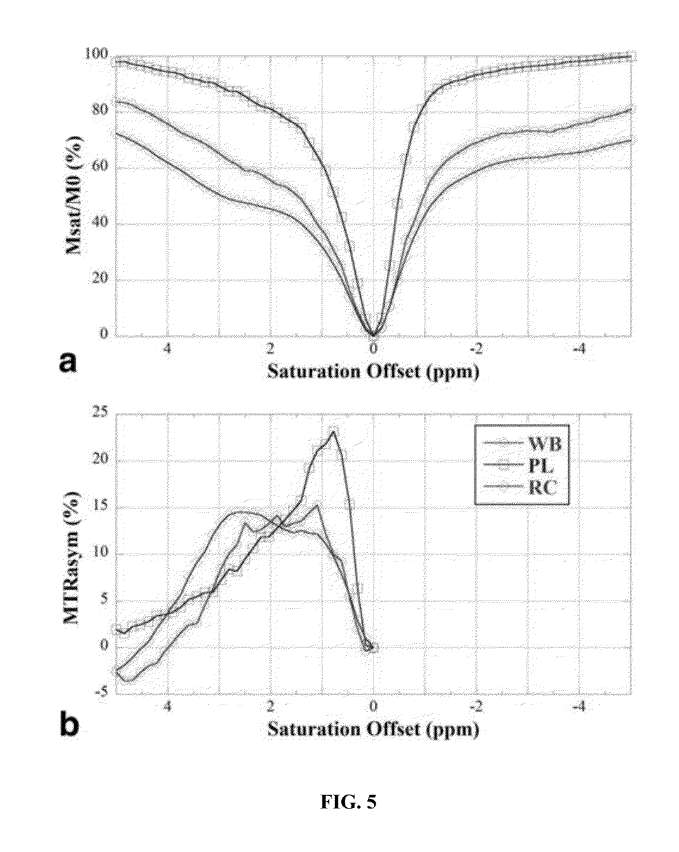

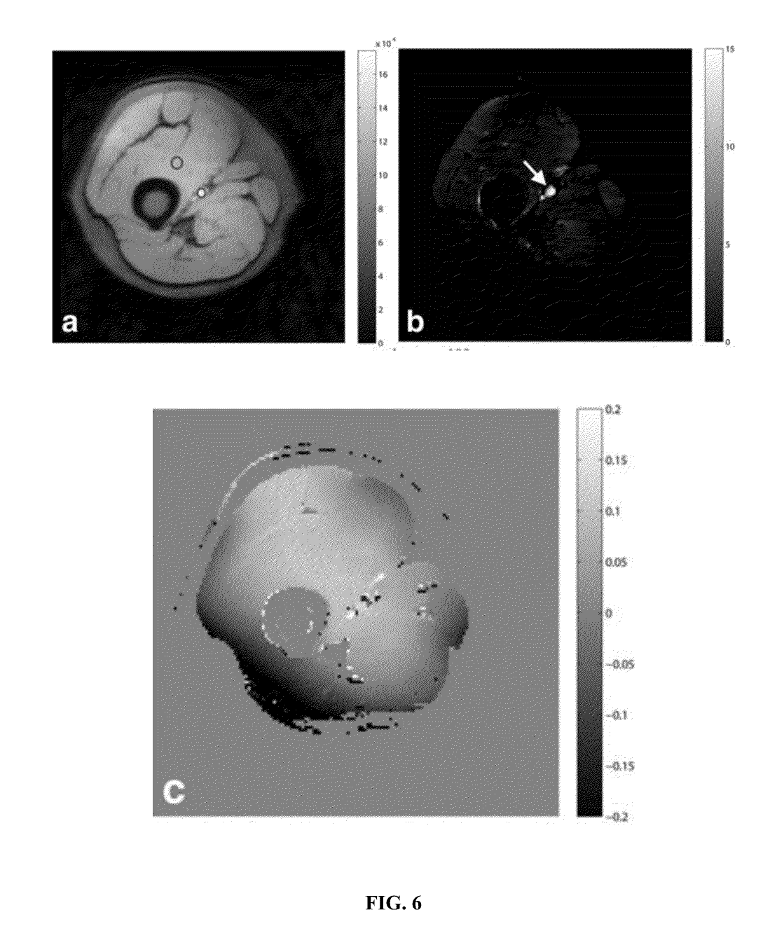

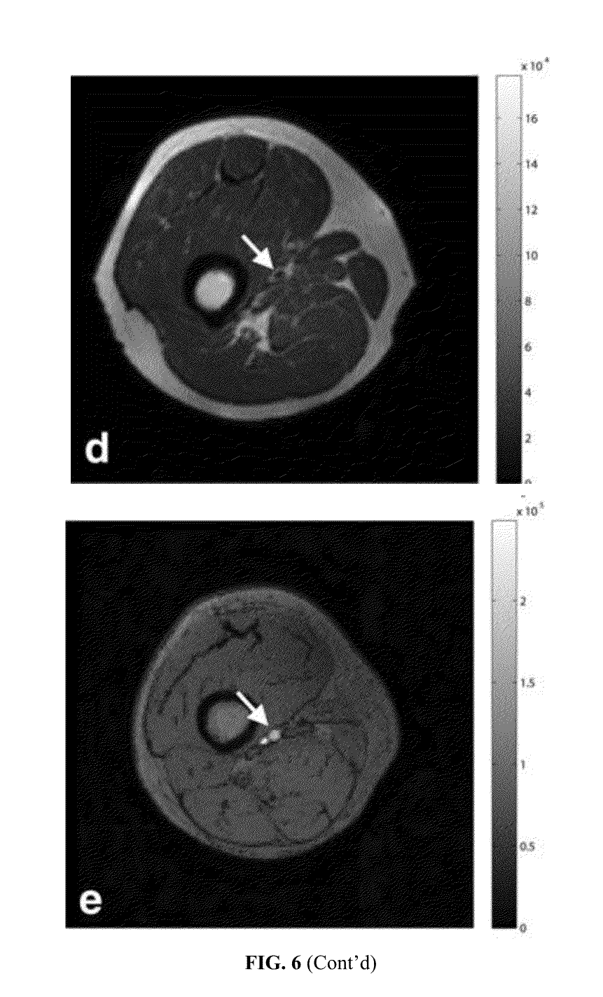

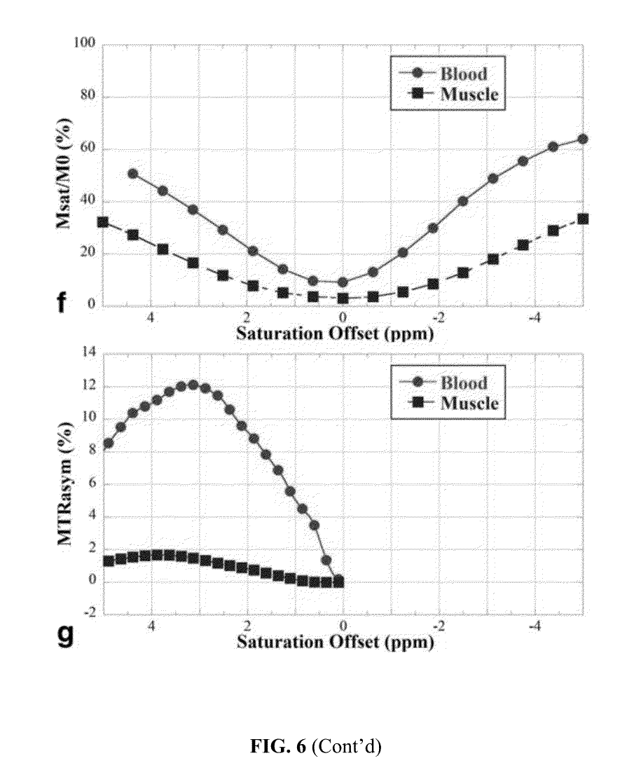

The proton exchange between these amide, amine protons, and water protons all contribute to the observed CEST effect in blood. This is also supported by the high concentration of amino acids in blood and both amide and amine protons have been reported as effective CEST endogenous agents. (Krebs 1950 Ann Rev Biochem. 19:409-430; Nicholson, et al. 1995 Anal Chem. 67:793-811.) For example, amide proton transfer (APT) imaging has shown to provide unique and complementary information for brain tumor imaging in animal models and humans studies. (Jones, et al. 2006 Magn Reson Med. 56:585-592; Zhou, et al. 2003 Nat Med. 9:1085-1090; Zhou, et al. 2003 Magn Reson Med. 50:1120-1126.) The CEST effect from the proton exchange between amine protons and water protons has been investigated as a potential technique to monitor the level of glutamate, which acts as a major neurotransmitter in the brain, and the level of proteins and peptides in brain. (Cai, et al. 2012 Nat Med. 18:302-306; Jin, et al. High field MR imaging of proteins and peptides based on the amine-water proton exchange effect. In: Proceedings of the 20th Annual Meeting of ISMRM, Melbourne, Australia 2012. p. 2339.) In addition to the amide and amine protons, hydroxyl protons (—OH) also contribute the CEST effect in blood, which can be seen in the MTR_{asym} plot of plasma in FIG. 5b. It has been reported that hydroxyl protons have a chemical shift of approximately 1 ppm downfield from bulk water and that the proton exchange rate of hydroxyl protons and water protons is in the order of 10³ s⁻¹. (Ling, et al. 2008 Proc Natl Acad Sci USA. 105:2266-2270; Schmitt, et al. 2011 Radiology. 260:257-264; Kim, et al. 2011 NMR Biomed. 24:1137-1144; Saar, et al. 2012 NMR Biomed. 25:255-261; van Zijl, et al. 2007 Proc Natl Acad Sci USA. 104:4359-4364; Liepinsh, et al. 1996 Magn Reson Med. 35:30-42.) It should be noted that the chemical exchange rate of amine and hydroxyl protons are considered to be in the medium to fast exchange regime (i.e. for a 3.0 T scanner, the chemical shift difference of 2.2 ppm is equivalent to Δf_{sw}=282 Hz or Δω_{sw}=1768 rad s⁻¹ and the chemical shift difference of 1 ppm is equivalent to Δf_{sw}=128 Hz or Δω_{sw}=804 rad s⁻¹; for 14.1 T spectrometer, the chemical shift difference of 2.2 ppm is equivalent to Δf_{sw}=1320 Hz or Δω_{sw}=8290 rad s⁻¹, and the chemical shift difference of 1 ppm is equivalent to Δf_{sw}=600 Hz or Δω_{sw}=3768 rad s⁻¹). Nevertheless, both amine and hydroxyl protons have been demonstrated to be suitable CEST agents. (Ling, et al. 2008 Proc Natl Acad Sci USA. 105:2266-2270; Schmitt, et al. 2011 Radiology. 260:257-264; Kim, et al. 2011 NMR Biomed. 24:1137-1144; Saar, et al. 2012 NMR Biomed. 25:255-261; van Zijl, et al. 2007 Proc Natl Acad Sci USA. 104:4359-4364; Cai, et al. 2012 Nat Med. 18:302-306; Liepinsh, et al. 1996 Magn Reson Med. 35:30-42; Jin, et al. High field MR imaging of proteins and peptides based on the amine-water proton exchange effect. In: Proceedings of the 20th Annual Meeting of ISMRM, Melbourne, Australia 2012. _{p.} 2339.)

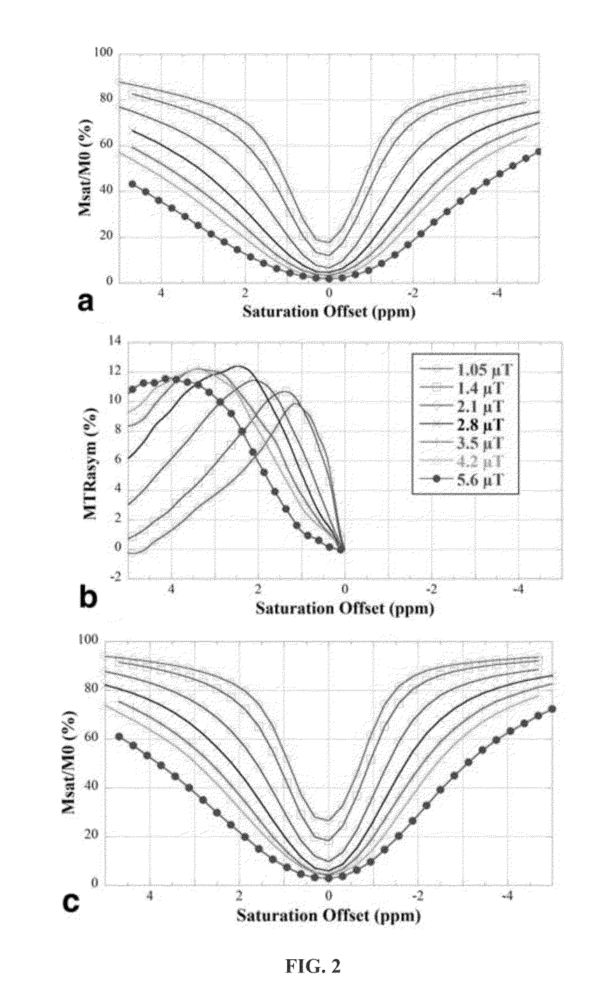

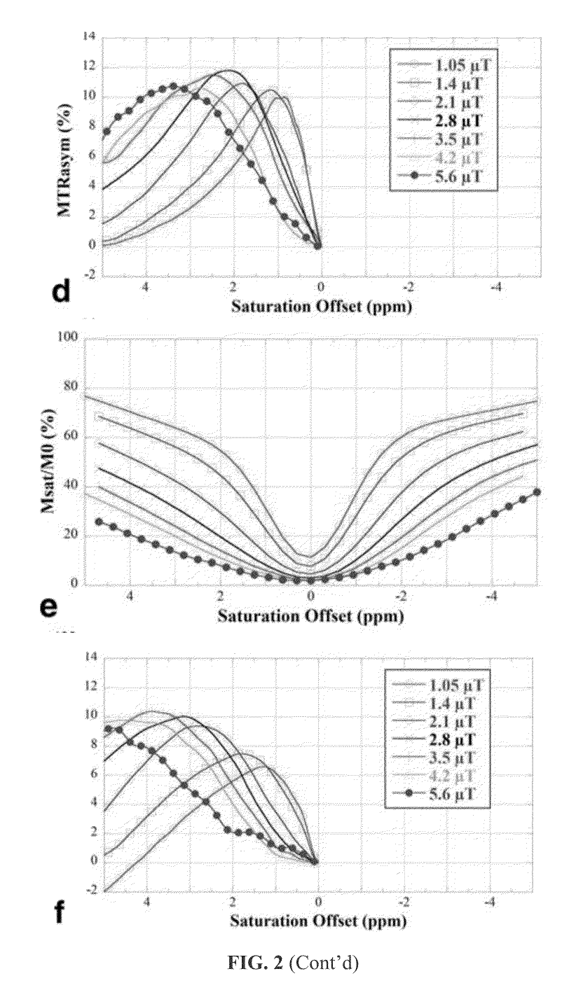

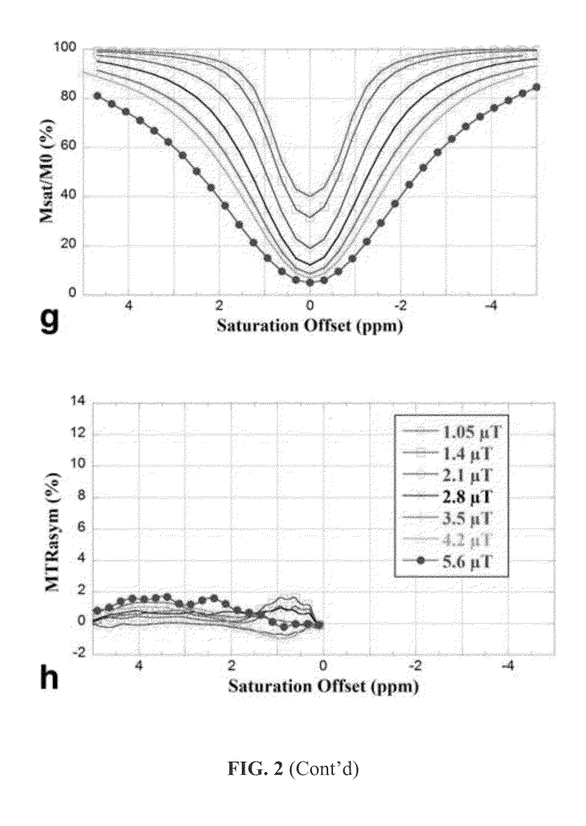

As to the other broad peak at −3.5 ppm relative to water resonance, it is clear that the protons in this area have an interaction with water protons. Because the protons in this area are from aliphatic components and have no chemical exchange with water protons, a possible mechanism for this interaction is the Nuclear Overhauser Enhancement (NOE) effect which is a type of cross-relaxation. (van Zijl, et al. 2011 Magn Reson Med. 65:927-948; van Zijl, et al. 2003 Magn Reson Med. 49:440-449.) In addition to the direct intramolecular NOE which occurs between different protons in the same macromolecules, “there are exchange-relayed intramolecular NOEs, in which” the saturation “is transferred from water to the molecules through rapidly exchangeable groups (mainly OH and NH2, but also NH) and subsequently to the backbone aliphatic protons”. (van Zijl, et al. 2011 Magn Reson Med. 65:927-948.) The phenomenon of proton MT from water to macromolecules in NMR as discussed above also holds for the opposite process, which corresponds to the CEST experiment. This explains the two shallow decays at 3 ppm and −3.5 ppm relative to water resonance in the Z-spectrum of red blood cells (FIG. 5a). However, the NOE effect seems not significant in plasma, which could reflect the difference of components and the dynamic process between red blood cells and plasma. Therefore, the MTR_{asym} analysis of blood is a combined result of the competitive effects of CEST in the downfield and NOE in the upfield. The negative MTR_{asym} values observed in the analyses performed on red blood cells (FIG. 2f and FIG. 5b) and whole blood (FIG. 5b), may be due to the relatively stronger NOE effect than the CEST effect in those specific experimental conditions.

The porcine blood experiments performed on a 3.0 T scanner showed that the maximum of MTR_{asym} value of approximately 12% at room temperature (and 10% at 37° C.) was achieved when blood was used as a CEST agent. The maximum MTR_{asym} values obtained with various power levels (FIG. 2) shifted from approximately 1 ppm to 3.5 ppm for plasma by changing the power lever from 1.05 μT to 5.6 μT and from 1.2 ppm to 5ppm for red blood cells. This phenomenon is likely due to the presence of multiple components in blood with different exchange rates that contribute to the observed MTR_{asym} effect as discussed above. (Krebs 1950 Ann Rev Biochem. 19:409-430; Nicholson, et al. 1995 Anal Chem. 67:793-811.) Sun et al showed that the proton transfer (PT) efficiency increased with the increase of RF power and decreased when the RF power was compatible to the resonance frequency difference between the solute and water peak. (Sun, et al. 2005 J Magn Reson. 175:193-200.) Their simulation also indicated that the PT efficiency increased with greater chemical exchange rates and the optimal power for different exchange rate varied. In the case of blood, the PT efficiency of exchangeable protons with a resonance frequency close to that of water, increases at a slower rate (or even decreases) as a result of increasing power than that of exchangeable protons further away from the water resonance, which could be a motive for the observed shift. Another possible reason for the shift of the maximum of the MTR_{asym} to a higher frequency with higher saturation power levels may be caused by the increased direct saturation that dominates over the CEST and MT effects. As a result, a reduced MTR_{asym} effect close to the water frequency is observed, resulting in a shift of the maximum MTR_{asym} value to a higher frequency. Thus, the complex CEST effect, or more accurately: the MTR_{asym} analysis, observed in blood sample results from the coexistence of multiple components, the chemical exchange effect and the NOE effect.

To observe the CEST effect of blood in human volunteers, single-slice 2D CEST imaging was performed on the femoral artery and the M1-segment of the MCA. MTR_{asym} analysis showed maximum values of approximately 12% for arterial blood at 3 ppm, which correlates with the results found using blood samples, whereas surrounding muscular and brain tissue showed maximum values of 2% and 3%, respectively.

It has been shown herein that blood is a feasible CEST agent that provides sufficient contrast relative to surrounding tissue. An application that takes advantage of blood as a CEST agent is MR angiography. Since the CEST signal in blood is determined by the concentration of exchangeable protons rather than the inflow of unsaturated protons, successful implementation of CEST can provide a angiography method (angioCEST) that is less sensitive to artifacts caused by slow or complex flow observed in aneurysms and stenoses, which have shown to cause misinterpretation of TOF or phase-contrast based methods [37-44]. (Bernstein, et al. 2001 Magn Reson Med. 46:955-962; Deutschmann, et al. 2007 AJNR Am J Neuroradiol. 28:628-634; Boulin, et al. 2001 Radiology. 219:108-113; Cottier, et al. 2003 AJNR Am J Neuroradiol. 24:1797-1803; Gibbs, et al. 2005 JMRI J Magn Reson Im. 21: 97-102; Jäger, et al. 2000 AJNR Am J Neuroradiol. 21:1900-1907; Kaufmann, et al. 2010 AJNR Am J Neuroradiol. 31:912-918; Okahara, et al. 2002 Stroke 33:1803-1808.)

Thus, in one aspect, the Invention generally relates to a method for imaging blood of a mammal, including human, in vivo. The method includes imaging the mammal using a CEST-based magnetic resonance imaging procedure wherein blood of the subject serves as the CEST contrast agent.

The method may be invasive or non-invasive. In certain preferred embodiments, the method is non-invasive.

The method may be utilized to image any organ or part of a mammal, including arteries, veins or heart chambers.

In certain preferred embodiments, the method is performed without the use of any exogenous CEST or other contrast agents. In certain embodiments, one or more exogenous CEST contrast agents are used for further enhancement of imaging contrast.

In another aspect, the invention generally relates to a method for performing angiography of a mammal, including human. The method includes performing CEST imaging with blood serving as the endogenous CEST contrast agent.

In certain embodiments, the angiography is the imaging of an artery of the subject. Any artery or a part or portion of an artery may be imaged, for example, an artery is selected from femoral artery, carotid artery, aorta, pulmonary artery, brachialis artery, hepatic artery, and iliac artery.

In certain embodiments, the angiography is the imaging of a vein of the mammal. Any vein or a part or portion of a vein may be imaged, for example, a vein selected from great saphenous vein, pulmonary veins, hepatic portal vein and hypophyseal portal system, and Thebesian veins.

In certain embodiments, the angiography is the imaging of one or more heart chambers of the mammal.

In certain embodiments, the angiography is the imaging of one or more parts of the brain of the mammal.

In certain preferred embodiments, the angiography is performed without any exogenous CEST contrast agent.

In yet another aspect, the invention generally relates a to method for diagnosing a mammal, including human, of a disease or condition. The method includes: performing CEST spectroscopy with the mammal's blood serving as the endogenous CEST contrast agent; and obtaining a magnetic resonance image of at least a part of a vascular system of the mammal.

In certain preferred embodiments, the diagnostic method is non-invasive.

In certain embodiments, the magnetic resonance image is an image of an artery of the subject, for example, selected from femoral artery, carotid artery, aorta, pulmonary artery, brachialis artery, hepatic artery, and iliac artery of the subject.

In certain embodiments, the magnetic resonance image is the image of a vein of the mammal, for example, selected from great saphenous vein, pulmonary veins, hepatic portal vein and hypophyseal portal system, and Thebesian veins.

In certain embodiments, the magnetic resonance image is the image of one or more heart chambers of the mammal. In certain embodiments, the magnetic resonance image is the image of one or more parts of the brain of the mammal.

In certain preferred embodiments, the CEST imaging is performed without any exogenous CEST contrast agent.

### EXAMPLES

To demonstrate that blood is a suitable CEST agent that generates sufficient CEST contrast relative to surrounding tissues, ex vivo and in vivo experiments are performed as provided hereinbelow.

### Porcine Blood Samples

Experiments were approved by the Institutional Animal Care and Use Committee of the University of Massachusetts Medical School. Arterial blood samples (30 ml per animal) were collected from three pigs that were used for an unrelated study. The mean weight was approximately 40 kg and the animals showed no evidence of any blood disease throughout the course of the studies. Whole blood samples were mixed (ratio 10:1) with Anticoagulant Citrate Dextrose (ACD) solution (Baxter healthcare Cor. North Carolina 28752) immediately after collection and analyzed using a handheld blood gas analyzer (i-STAT, Abbott, Princeton, N.J.) (pH: 7.36±0.08; Hct(%): 25.5±2.2; Hb (g/dL): 8.7±0.7; sO₂(%): 99.5±0.7). A part of each whole blood sample was centrifuged (Centrifuge 5804R, Eppendorf AG, Hamburg, Germany) with 2000 rcf for 10 minutes to separate plasma from red and white blood cells and platelets. Whole blood, plasma, red blood cells, and ACD were placed in separated 10 mm vials and immersed into a copper sulfate solution for MR-imaging. In addition, 5 mm tubes were filled with blood samples for NMR spectroscopy.

Because temperature influences the chemical exchange rate and may therefore have an impact on the CEST effect, CEST MRI experiments were performed at a physiological temperature of 37±1° C. and were compared to experiments performed at room temperature. The temperature was controlled by a monitoring & gating system (Model 1025, SA Instruments, Inc. Stony Brook, N.Y.). Unless indicated otherwise, all MRI and NMR experiments were performed at room temperature.

### Human Subjects

In order to provide a proof-of-concept, CEST imaging was performed on 6 healthy volunteers (3 males and 3 females; mean age, 23 years; age range, 20-30 years). This study was approved by the Institutional Review Board of the University of Massachusetts Medical School. All participants signed consent form and none of the participants had any contraindications to MR imaging. For three volunteers MR data was acquired on the right upper leg with the objective of generating sufficient CEST contrast from the femoral artery relative to the surrounding tissue. To evaluate the feasibility of CEST imaging in the brain, MR-imaging was performed on the M1-segment of the middle cerebral artery (MCA) of the remaining three volunteers.

### MRI Acquisition

All MRI experiments were performed on a 3.0 T (128 MHz for 1H) Achieva whole-body MR scanner (Philips Medical Systems, Best, the Netherlands).

For the ex vivo blood phantom study, a custom-made solenoid T/R coil with a diameter of 75 mm was used. To account for the sensitivity of CEST to B0 inhomogeneities, Water Saturation Shift Reference (WASSR) technique was used to compensate the potential variation of B0. The pulse sequences of WASSR and CEST are essentially the same. Both consist of a rectangular continuous-wave (CW) RF pre-saturation pulse followed by a Turbo Spin Echo (TSE) imaging pulse sequence. However, a much lower power and shorter duration of pre-saturation pulse was used for WASSR, which was sufficient to obtain direct saturation while minimizing the CEST and magnetization transfer (MT) effects. In this study, a 0.7 μT pulse of 50 ms was used for WASSR pre-saturation. Thirty-three dynamic experiments were performed during phantom WASSR scan by changing the offset frequency from 32 Hz to −32 Hz relative to bulk water on-resonance frequency with a step size of 2 Hz. CEST data was acquired using different pre-saturation power (1.05 μT, 1.4 μT, 2.1 μT, 2.8 μT, 3.5 μT, 4.2 μT and 5.6 μT) with the duration of 1000ms. The frequency of the pre-saturation pulse was varied from 640 Hz (5 ppm) to −640 Hz (−5 ppm) with a step size of 40 Hz, to generate a Z-spectrum. A full preparation phase, including RF power optimization, resonance frequency determination, second order shimming, and receiver gain optimization were performed before WASSR data acquisition. The experimental conditions were kept constant throughout the WASSR and CEST experiments. Image data was acquired using a single slice parallel to the short axis of the cylindrical tube with a slice thickness of 5 mm and a field of view (FOV) of 50 mm×50 mm with matrix size of 168×160 (TR/TE=3000/10 ms, flip angle (FA)=90°, TSE-factor=16, low-high scan order, NSA=1).

For the imaging experiments on the human subjects, the whole-body transmitter coil was used to transmit the RF pre-saturation power to ensure that pre-saturation is achieved inside and outside the imaging volume to avoid non-saturated blood from flowing into the field of view. Subsequently, images were acquired by using an 8-element receive-only SENSE knee or head coil. Turbo Field Echo (TFE) pulse sequence instead of a TSE pulse sequence was used to avoid the void effect of the moving blood in TSE pulse sequence. 3D TOF angiography imaging of the right upper leg and brain were performed to localize the geometry of interest (SENSE factor=2; TR/TE=25/3.5 ms, FA=20°, NSA=1). Subsequently, WASSR and CEST data were acquired for the same axial slice positioned at the right upper leg containing the femoral artery, and the M1-segment of the MCA. WASSR data was obtained by using a 0.7 μT pre-saturation pulse with 50 ms duration. A total of 33 dynamics were acquired while varying the pre-saturation frequency from 64 Hz to p31 64 Hz with a step size of 4 Hz. CEST imaging was done by using a 3.5 μT pre-saturation pulse with 500 ms duration. A total of 17 dynamics were acquired while varying the pre-saturation frequency from 640 Hz to −640 Hz with a step size of 80 Hz. Single slice WASSR and CEST images were acquired by using a TFE with a slice thickness of 5 mm, a FOV of 180 mm×180 mm, and matrix size of 128×110 for leg imaging (TFE factor=29, SENSE factor=2; shot interval=3000 ms; TR/TE=7.1/4.2 ms, FA=20°, NSA=2 and 6 for WASSR and CEST, respectively). The parameters for brain imaging were the same as those used for the leg, except for the geometric parameters (e.g. slice thickness of 2 mm, FOV of 230 mm×230 mm, and matrix size of 152×123). MR parameters for WASSR and CEST were summarized in Table 1.

TABLE 1
WASSR and CEST imaging parameters

| | | | | Offset | Offset | Number | | |
|---|---|---|---|---|---|---|---|---|
| | | Duration | Power | Range | Gap | of | Imaging | |
| | | (ms) | (μT) | (Hz) | (Hz) | Offsets | Method | NSA |
| Blood | WASSR | 50 | 0.7 | 32 to −32 | 2 | 33 | TSE | 1 |
| Phantom | CEST | 1000 | 1.05, 1.4, | 640 to −640 | 40 | 33 | TSE | 1 |
| | | | 2.1, 2.8, | | | | | |
| | | | 3.5, 4.2, | | | | | |
| | | | 5.6 | | | | | |
| Human | WASSR | 50 | 0.7 | 64 to −64 | 4 | 33 | TFE | 2 |
| (Leg and | CEST | 500 | 3.5 | 640 to −640 | 8 | 17 | TFE | 6 |
| Brain) | | | | | | | | |

To further study the CEST effect in blood, high-resolution 1H NMR spectroscopy and Z-spectrum were performed by using a 14.1 T (600 MHz for 1H) Varian Unity Inova spectrometer equipped with a 5-mm triple resonance probe. Spectroscopy data was acquired from whole blood, plasma and red blood cells at room temperature. To identify the possible proton exchange between water and the exchangeable solute labile protons qualitatively, both spectra with and without water pre-saturation were acquired. To decrease the radiation damping effect, a flip angle of 5 degrees was used for all samples without water saturation and 45 degrees for all samples with water saturation. The repetition time used was 11 seconds and a line broadening of 1 Hz was used for Fourier transformation. The Z-spectra were acquired with pre-saturation duration of 3 seconds, power of 2.8 μT, and 65 off-resonance steps with the frequency step of 93.75 Hz.

### Data Analysis

MR-imaging data were transferred to a PC and processed by using in-house developed software developed in Matlab (MathWorks, Natick, Mass.). Regions of interest (ROIs) were manually drawn in the WASSR data and were automatically copied to the corresponding CEST data. For each ROI, mean intensity per dynamic was calculated to enhance the SNR and the saturated signal (Sₛₐₜ) was normalized by the unsaturated signal (S₀) to generate Z-spectra. WASSR shift per ROI was calculated as described by Kim et al. (Kim, et al. 2009 Magn Reson Med. 61:1441-1450.) Resulting WASSR shift was applied to the corresponding CEST data after reducing signal noise by using a moving average filter with a kernel size of 5.

Z-spectra were generated by plotting the normalized signal (Sₛₐₜ/S₀) as a function of the irradiation RF-frequency relative to the water frequency (Δω), which was assigned to 0 ppm. Magnetization Transfer Ratio asymmetry (MTR_{asym}) analyses [3, 4] were performed by utilizing the asymmetry of the Z-spectrum:

MTR_{asym}(Δω)=(Sₛₐₜ(−Δω)−Sₛₐₜ(Δω))/S₀. (1)

MRI Results from Porcine Blood Samples

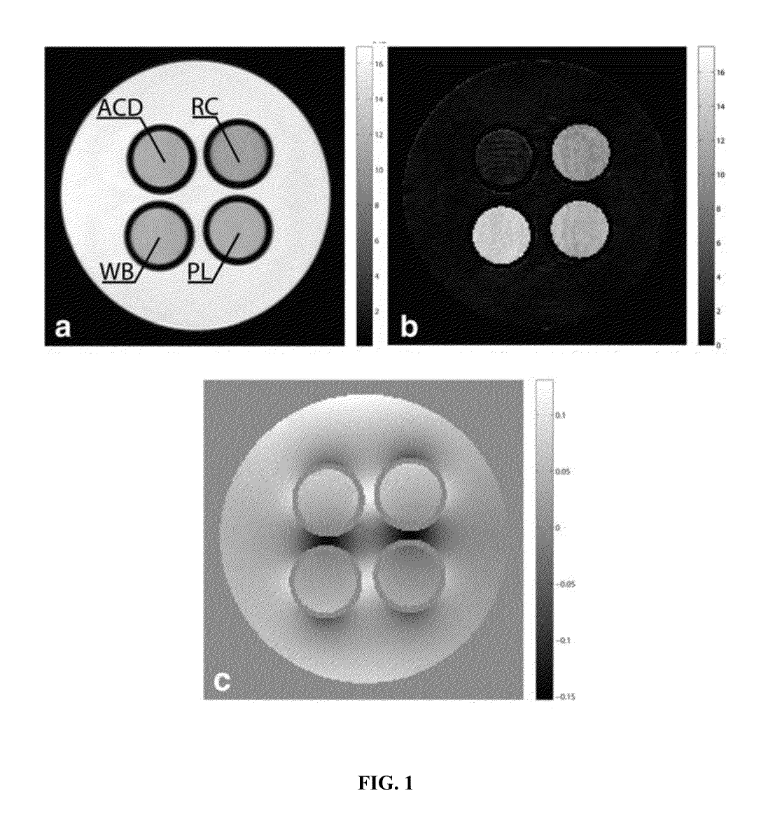

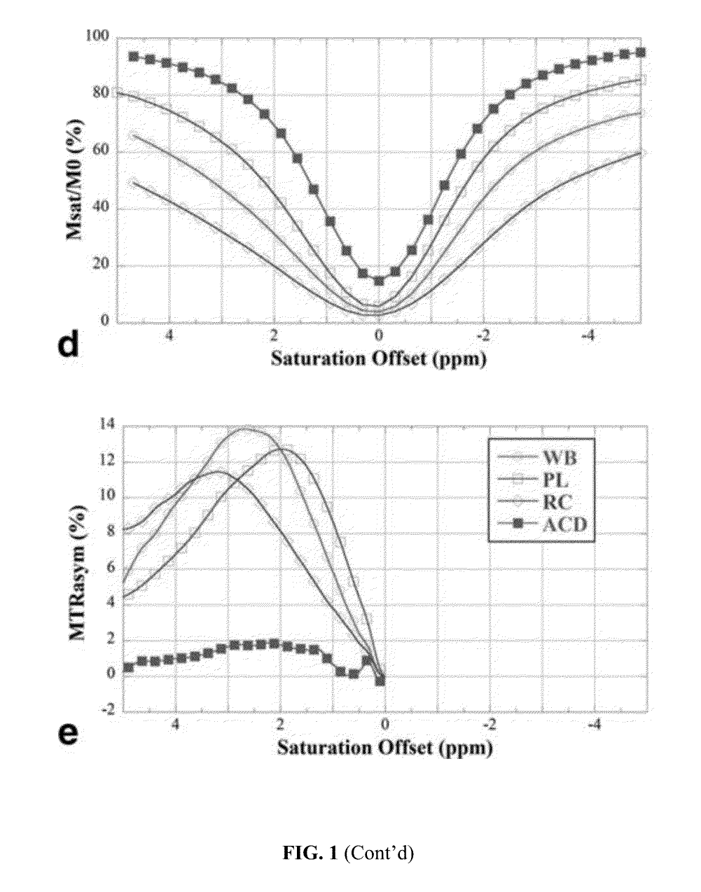

In FIG. 1, a representative proton density image (TR/TE=3000 ms/10 ms) (1a), the corresponding CEST MTR_{asym} image (1b) and WASSR shift map (1c) are given of four of the cylindrical tubes with porcine whole blood (WB), plasma (PL), red blood cells (RC) and ACD emerged in a beaker containing a copper sulfate solution at room temperature. FIGS. 1d and 1e show the corresponding Z-spectra and MTR_{asym} graphs, respectively. The CEST MTR_{asym} image was generated by calculating the MTR_{asym} value for all pixels using a fixed pre-saturation offset of 2.5 ppm and a pixel-wise implementation of the WASSR and CEST data processing. The pre-saturation power used was 2.8 μT and the pulse duration was 1000 ms. The CEST MTR_{asym} image shows clear contrast of blood relative to the surrounding copper sulfate solution and ACD. The measured MTR_{asym} values of porcine whole blood, plasma, red blood cells, and ACD were 13.8±0.4, 11.9±0.6, 10.1±0.7, and 1.7±0.6, respectively.

FIGS. 2a-h show the typical Z-spectra and corresponding MTR_{asym} graphs for porcine whole blood (2a-b), plasma (2c-d), red blood cells (2e-f), and ACD (2g-h), as a function of different pre-saturation power levels. From these graphs, several distinct features can be identified. (1) A significant CEST effect with a maximum MTR_{asym} value of approximately 12% can be observed for whole blood and plasma, and 10% for red blood cells. For the various samples used in these experiments, the maximum MTR_{asym} value ranged from 10% to 15%. However, ACD shows no significant CEST effect relative to blood samples, which proves that the CEST effect in blood sample can be ascribed to blood and not the anticoagulant. (2) MTR_{asym} graphs show that blood has a broad CEST effect ranging from approximately 0 to 5 ppm in this study. (3) The Z-spectra of red blood cells are expansive as compared to the corresponding plasma. This phenomenon was likely caused by the fact that red blood cells have a short T2 value relative to plasma, which results in a broader direct water saturation profile. The measured T2 value of whole blood, plasma, and red blood cells were approximately 170 ms, 316 ms and 92 ms, respectively. The wider Z-spectra curve of red blood cells impacts the MTR_{asym} curve relative to plasma, however the maximum MTR_{asym} value of red blood cells and plasma are similar. (4) It can be observed that the maximum of the MTR_{asym} plot was shifted from approximately 1 ppm to 3.5 ppm for plasma by changing the power level from 1.05 μT to 5.6 μT, and from 1.2 ppm to 5 ppm for red blood cells. The maximum of the MTR_{asym} value increases first and then decreases slightly when the pre-saturation power is increased and simultaneously shifts to higher saturation offsets. This phenomenon is more significant for red blood cells than for plasma. (5) FIG. 2 shows that negative MTR_{asym} values can be seen at saturation offset frequencies over 4.3 ppm and at low saturation power of 1.05 μT in red blood cells.

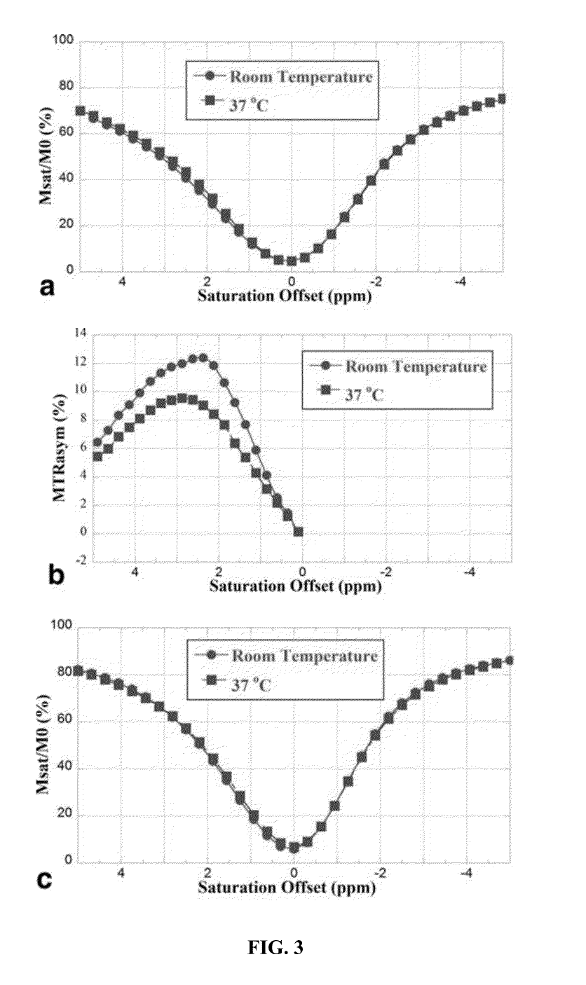

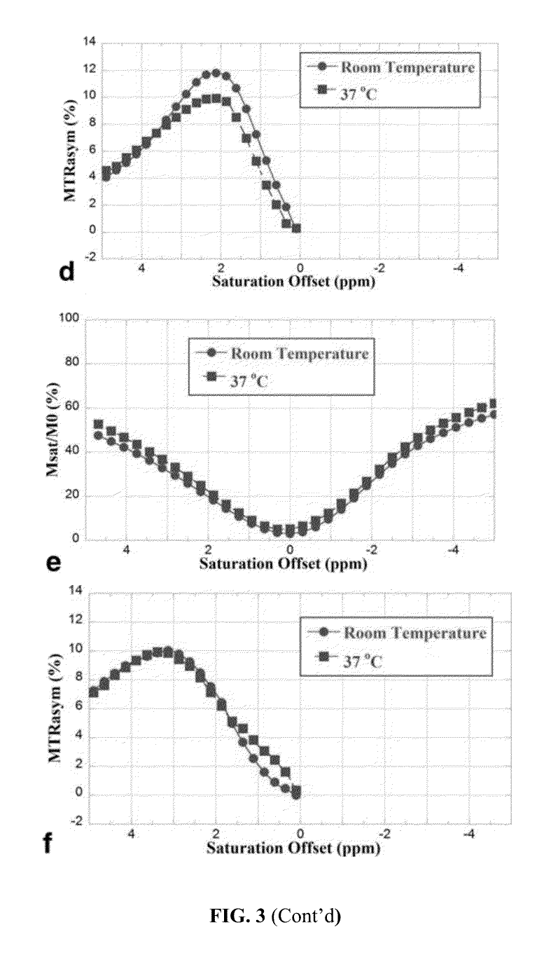

FIGS. 3a-f compare the Z-spectra and the corresponding MTR_{asym} for the porcine whole blood (3a-b), plasma (3c-d), red blood cells (3e-f) at room temperature and physiological temperature of 37° C. The pre-saturation power used was 2.8 μT and the pulse duration was 1000 ms. From FIG. 3, it can be seen that the maximum MTR_{asym} value of plasma decreases approximately 2% at higher temperature while that of red blood cells only shows a small deviation. In addition, a shift of 0.5 ppm was observed for whole blood. This result demonstrates that CEST experiments performed at room temperature are representative of experiments performed at physiological temperature when a 2% decrease is taken into account.

NMR Results from Porcine Blood Samples

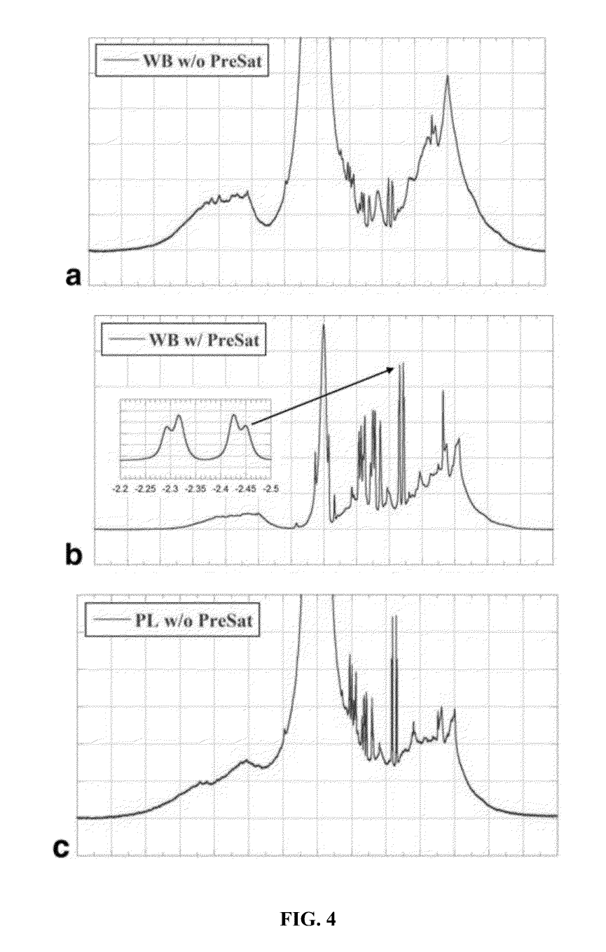

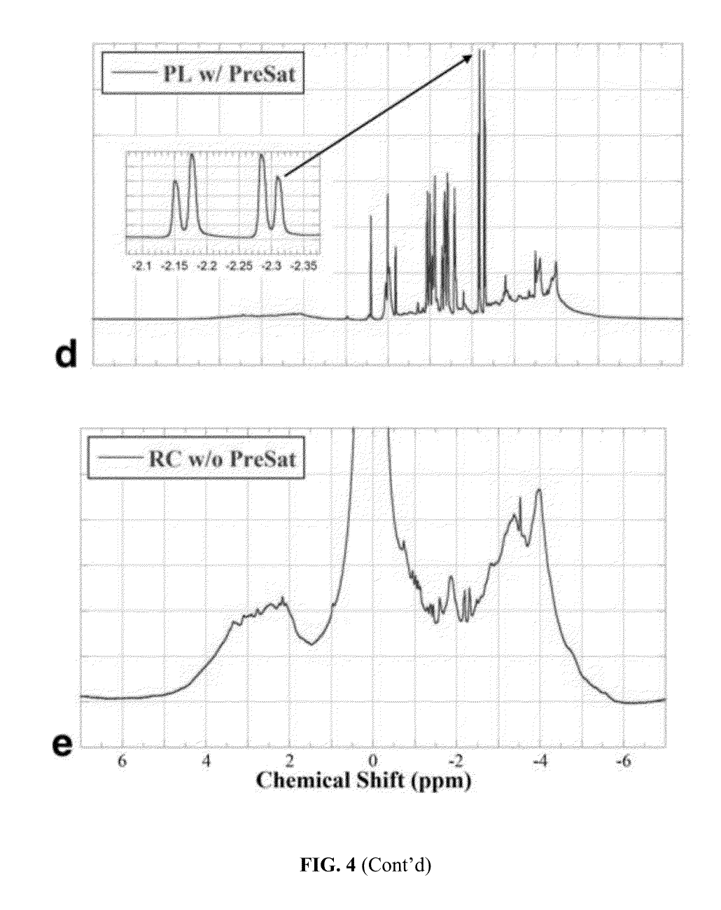

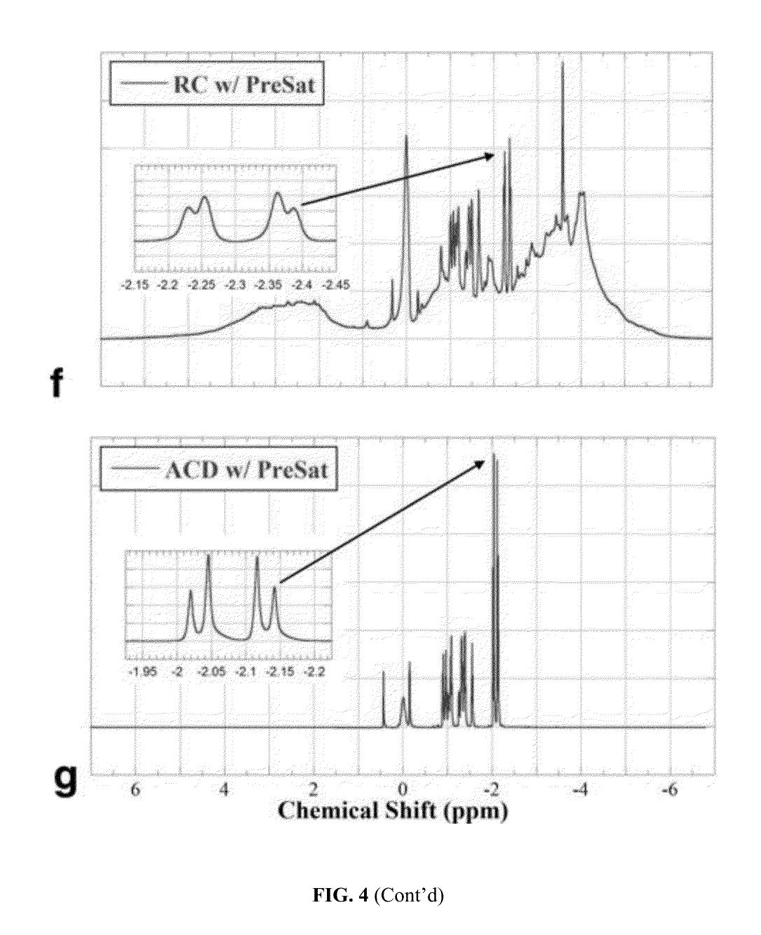

All NMR experiments were performed on 14.1 T NMR spectrometer. FIG. 4 shows the results of the NMR experiments performed on whole blood (4a-b), plasma (4c-d), and red blood cells (4e-f). All samples were done without (a, c, e) and with water pre-saturation (b, d, O. The NMR result of ACD (4g) with water pre-saturation was provided for comparison. The Z-spectrum and MTR_{asym} analysis of blood samples obtained with NMR spectroscopy are given in FIGS. 5a and b, respectively.

Similar to the results at 3.0 T, some distinct features can be identified. (1) There are two broad NMR peaks for both plasma and red blood cells samples; one is centered approximately at 3 ppm and roughly ranged from 1.5 (or smaller) to 4.5 ppm and the other is centered approximately at −3.5 ppm and roughly ranged from −2.0 to −6.0 ppm relative to water. (2) The intensity of these two broad peaks decreases greatly relative to the four peaks seen around at −2.1 ppm relative to water (which corresponding to 2.7 ppm for normal NMR spectroscopy, see insert). These four peaks are from the —CH₂ protons of citric acid [24] of ACD and have no proton exchange with water protons. The possible reason for the small difference in chemical shift of —CH₂ in the blood sample and that in ACD is the slight deviation during manual assignment of water peak, which was assigned to the highest point of residual water peak. Another potential reason for this difference in chemical shift may be caused by the solvent that was used. This decrease demonstrates that the protons corresponding to these two broad peaks have interaction with water protons; the main possible interactions could be chemical exchange and dipolar-dipolar interaction. (3) The Z-spectra of plasma and red blood cells show strong asymmetry. Clear signal decays can be seen at 3 ppm and −3.5 ppm relative to bulk water, which is consistent with the NMR findings. This confirms that the protons corresponding to the two broad peaks centered at 3 ppm and −3.5 ppm interact with water protons. (4) The asymmetry of Z-spectra of plasma and red blood cells can be seen clearly in the MTR_{asym} analysis. Similar to the results on a 3.0 T MRI scanner, MTR_{asym} graphs acquired on a 14.1 T spectrometer show broad MTR_{asym} peak ranging from approximately 0 to 4 ppm. (5) The shape of MTR_{asym} graph of plasma is different from that of red blood cells. The maximum of the MTR_{asym} plot of plasma is close to 0.75 ppm relative to water frequency, and 2.5 ppm for red blood cells.

Results from Human Subjects

FIGS. 6a-e show an axial slice of typical MR-imaging data obtained from the leg of a volunteer. FIG. 6a shows an anatomical image with two ROIs corresponding to arterial blood (red) and muscle (blue). Matching CEST MTR_{asym} image at 3 ppm (FIG. 6b) demonstrates clear enhancement of the femoral artery (arrows). The WASSR shift map used to calculate the CEST MTR_{asym} image is given in FIG. 6c. Images shown in FIGS. 6d and 6e are the T2w-TSE and the TOF data, respectively. The graphs in FIGS. 6f and 6g are the Z-spectra and MTR_{asym} corresponding to the ROIs in FIG. 6a. A maximum MTR_{asym} value of about 12% for blood is observed, which is similar to the value found using the porcine blood samples. The maximum MTR_{asym} value found in muscle was approximately 2%.

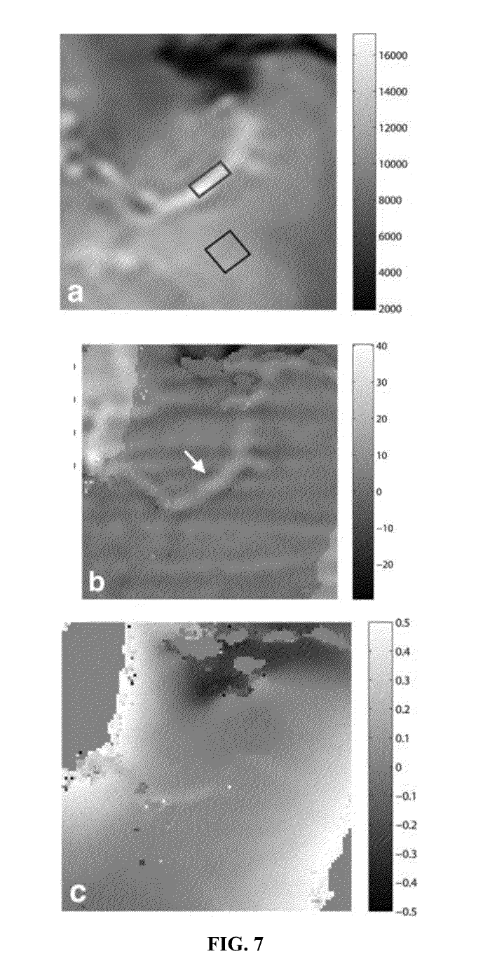

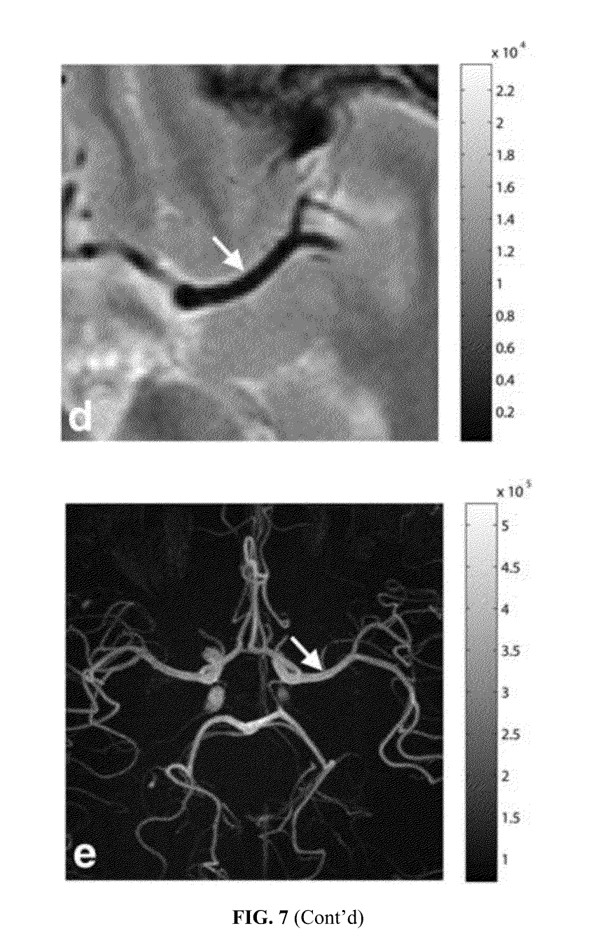

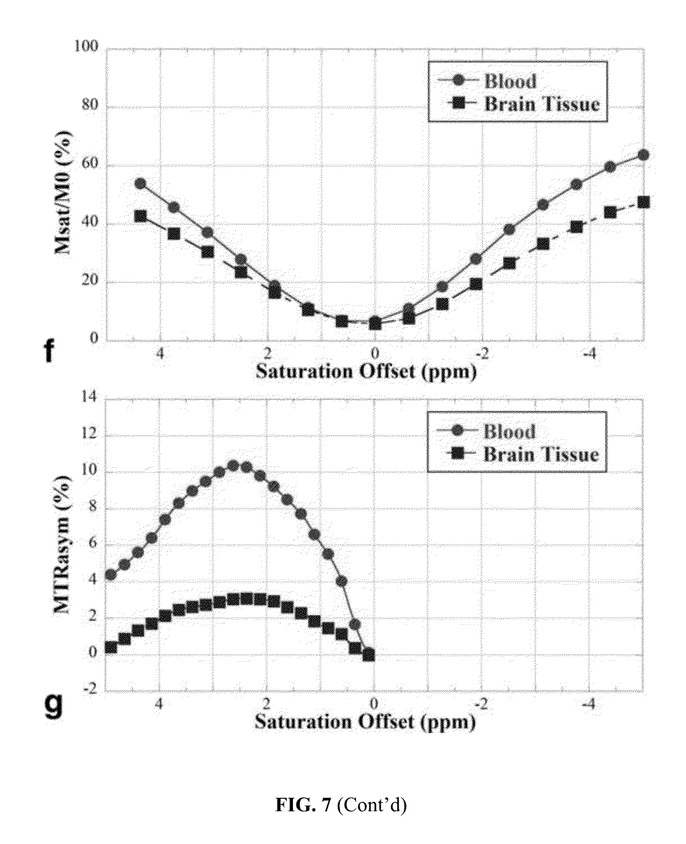

Similar results were demonstrated in the M1-segment of the MCA (FIGS. 7a-g). A maximum MTR_{asym} value of approximately 10% for blood was observed, compared to surrounding brain tissue which only generated maximum MTR_{asym} values of approximately 3%.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A method for imaging blood of a mammal, including human, in vivo comprising imaging the mammal using a CEST-based magnetic resonance imaging procedure wherein blood of the subject serves as the CEST contrast agent.

2. The method of claim 1, wherein the method is non-invasive.

3. The method of claim 1, wherein the method images an artery, vein or heart chamber of the mammal.

4. The method of claim 1, wherein one or more exogenous CEST contrast agents are used for further enhancement of imaging contrast.

5. The method of claim 1, wherein no exogenous CEST contrast agent is used.

6. A method for performing angiography of a mammal, including human, comprising performing CEST imaging with blood serving as the endogenous CEST contrast agent.

7. The method of claim 6, wherein the angiography is the imaging of an artery of the subject.

8. The method of claim 7, wherein the artery is selected from femoral artery, carotid artery, aorta, pulmonary artery, brachialis artery, hepatic artery, and iliac artery.

9. The method of claim 6, wherein the angiography is the imaging of a vein of the mammal.

10. The method of claim 9, wherein the vein is selected from great saphenous vein, pulmonary veins, hepatic portal vein and hypophyseal portal system, and Thebesian veins.

11. The method of claim 6, wherein the angiography is the imaging of one or more heart chambers of the mammal.

12. The method of claim 6, wherein the angiography is the imaging of one or more parts of the brain of the mammal.

13. The method of claim 6, wherein the angiography is performed without any exogenous CEST contrast agent.

14. A method for diagnosing a mammal, including human, of a disease or condition, comprising:
performing CEST imaging with the mammal's blood serving as the endogenous CEST contrast agent; and
obtaining a magnetic resonance image of at least a part of a vascular system of the mammal.

15. The method of claim 14, wherein the method is non-invasive.

16. The method of claim 14, wherein the magnetic resonance image is an image of an artery of the subject.

17. The method of claim 16, wherein the artery is selected from femoral artery, carotid artery, aorta, pulmonary artery, brachialis artery, hepatic artery, and iliac artery.

18. The method of claim 14, wherein the magnetic resonance image is the image of a vein of the mammal.

19. The method of claim 18, wherein the vein is selected from great saphenous vein, pulmonary veins, hepatic portal vein and hypophyseal portal system, and Thebesian veins.

20. The method of claim 14, wherein the magnetic resonance image is the image of one or more heart chambers of the mammal.

21. The method of claim 14, wherein the magnetic resonance image is the image of one or more parts of the brain of the mammal.

22. The method of claim 14, wherein the CEST imaging is performed without any exogenous CEST contrast agent.
